(19) United States
(12) Patent Application Publication
(10) Pub. No.: US 2013/0183357 A1
(43) Pub. Date: Jul. 18, 2013

(54) OXIDATIVELY CROSS-LINKED PROTEIN-BASED ENCAPSULATES

(71) Applicants: Aart Cornelis Alting, Ede (NL); Theodorus Arnoldus Gerardus Floris, Arnhem (NL); Fanny Chantal Jacqueline Weinbreck, Amersfoort (NL); Jeroen Grandia, Ede (NL); Freddie Van De Velde, HJ Ede (NL); Igor Bodnár, Utrecht (NL)

(72) Inventors: Aart Cornelis Alting, Ede (NL); Theodorus Arnoldus Gerardus Floris, Arnhem (NL); Fanny Chantal Jacqueline Weinbreck, Amersfoort (NL); Jeroen Grandia, Ede (NL); Freddie Van De Velde, HJ Ede (NL); Igor Bodnár, Utrecht (NL)

(73) Assignee: Nizo Food Research B.V.

(21) Appl. No.: 12/601,114

(22) PCT Filed: May 21, 2008
(86) PCT No.: PCT/NL08/50299

§ 371 Date: Apr. 12, 2010

(30) Foreign Application Priority Data
Nov. 29, 2007 (EP) 07121885.3

(51) Int. Cl. A23L 1/00; A61K 8/11; A61Q 19/00; A61K 9/50

(52) U.S. Cl. CPC A23L 1/0029; A61K 9/5089; A61K 9/5052; A61K 8/11; A61Q 19/00
USPC 424/401; 424/491; 424/94.1; 424/93.1; 514/1.1; 514/558; 514/731; 427/2.14; 426/89; 426/61; 426/590; 426/648; 426/650; 426/250; 426/311; 435/68.1

(57) ABSTRACT

The present invention provides a method of producing a protein-based encapsulate, said method comprising: providing an aqueous solution of a protein that is capable of forming disulfide cross-links; submitting said aqueous solution to a protein activation treatment to produce an aqueous suspension of activated protein aggregates, said suspension having a reactivity of at least 5.0 μmol thiol groups per gram of activated protein aggregates as determined in the Ellman's assay; dispensing said aqueous suspension in a gas or a water-immiscible liquid to produce suspension droplets having a diameter of 0.1-500 μm; and forming disulfide cross-links between the activated protein aggregates by contacting the activated protein aggregates with an oxidizing agent, optionally after said activated protein aggregates have been partially cross-linked by forming disulfide cross-links by means of heat treatment or by pressurization to a pressure in excess of 50 MPa. The aforementioned method offers the advantage that the characteristics of the protein-based encapsulation matrix can be controlled effectively. Furthermore, said method enables the preparation of protein-based encapsulates that very effectively protect the encapsulated components, e.g. against oxidation or moisture.

### FIELD OF THE INVENTION

The present invention concerns a method for making cross-linked protein-based encapsulates. These encapsulates may suitably be used to encapsulate a component to protect it from environmental factors that might otherwise deteriorate the quality thereof or to control the release of said encapsulated component. The encapsulates thus provided are suitable ingredients for various products, in particular food products.

### BACKGROUND OF THE INVENTION

The use of encapsulated ingredients in various products is widely known. In particular encapsulation techniques have been developed to protect ingredients that are to be applied in e.g. foodstuffs, beverages, nutritional supplements, cosmetic products, pharmaceutical products or animal feed. To this end encapsulation agents have been developed to meet the criteria of successfully providing long term stability and protection against deteriorating factors.

Acceptable encapsulating agents must be safe and non-hazardous to the consumer's health. For food products it should have a bland or no flavor. Besides protecting the encapsulated product from external factors such as oxygen, water, light or other compounds possibly causing deterioration, it should delay the release of an active ingredient pending its use.

Suitable encapsulation agents for food applications include natural gums, carbohydrates, fats and waxes and some proteins. Whereas gum Arabic is one of the most widely used encapsulation agent in food applications the use of proteins is limited. The main protein that has been evaluated for encapsulation is gelatin. Gelatin has been successfully applied as encapsulation agent in the pharmaceutical industry. However, due to the high viscosity of aqueous gelatin solutions, gelatin has limited use in spray-drying processes.

U.S. Pat. No. 5,601,760 describes a method for micro-encapsulation of a volatile or a non-volatile core material in an encapsulation agent consisting essentially of a whey protein. It is de scribed that whey protein isolate and whey protein concentrate, optionally in combination with milk-derived or non-milk derived carbohydrates, and also β-lactoglobulin and mixtures of β-lactoglobulin and α-lactalbumin were used in a spray-drying encapsulation process. The resulting encapsulates were said to protect the core against deterioration by oxygen or from detrimental of other compounds or materials, to limit the evaporation or losses of volatile core materials and to release the core upon full hydration reconstitution. One example describes encapsulation of anhydrous milk fat in whey protein isolate that has been heated at 80° C. for 30 minutes. This treatment results in denaturation of whey proteins.

EP-A 1 042 960 describes a cappuccino creamer with advantageous foaming properties. The creamer is prepared by spray-drying a slurry that includes as essential constituents protein, lipid and carrier. The lipid includes dairy fats and vegetable oils. Suitable carriers include gum Arabic and water soluble carbohydrates such as maltodextrin and lactose. The protein is partly denatured whey protein (concentrate or isolate). The product is said to contain buoyant, hydrated, insoluble, non-colloidal, irregularly shaped whey protein particles of approximately 10-200 microns in size, with an average particle size of about 60 microns. To provide coffee whitening and creamy mouth feel a significant amount of encapsulated fat has to be included.

U.S. Pat. No. 6,841,181 B2 describes the encapsulation of active food components using spray-drying technology. The process consists of mixing active ingredients with non-activated proteins and polysaccharides which are spray-dried to form a capsule. The capsules are 1-200 μm and up to 90% core material.

It is an object of the invention to provide a method for producing protein-based encapsulates, wherein the characteristics of the protein-based encapsulation matrix can be controlled effectively. Furthermore, the present invention aims to provide an encapsulation method that enables the preparation of protein-based encapsulates that very effectively protect the encapsulated components, e.g. against oxidation or moisture.

### SUMMARY OF THE INVENTION

The inventors have discovered that the aforementioned requirements can be fulfilled by an encapsulation method that employs the following steps:
providing an aqueous solution of a protein that is capable of forming disulfide cross-links;
submitting said aqueous solution to a protein activation treatment to produce an aqueous suspension of activated protein aggregates;
dispensing said aqueous suspension in a gas or a water-immiscible liquid to produce droplets having a volume weighted average diameter in the range of 0.1-500 μm; and
forming disulfide cross-links between the activated protein aggregates by contacting the activated protein aggregates with an oxidizing agent.

The activation step in the aforementioned process is a special form of protein denaturation and is crucial for the formation of disulphide cross-links between activated protein aggregates during the drying step. In the present method the activated protein aggregates are formed by irreversible denaturation of dissolved protein molecules, resulting in exposure of thiol groups that have the ability and accessibility to form disulfide bridges. In the course of the activation process, the reactive thiol groups of denatured protein molecules react together to form disulfide bridges. Thus, aggregates comprising a multitude of cross-linked protein molecules are formed. In the present method it is crucial that these aggregates retain reactive thiol groups as these reactive thiol groups are required for the cross-linking of the activated aggregates.

Not only cystein residues that have free thiol groups can participate in these cross-linking reactions, but also cystein residues that together form a disulfide bridge can react with a thiol group under the formation of a new disulfide bridge and the release of another free thiol group. This is why β-lactoglobulin can suitably be used as a cross-linkable protein even though this protein normally contains two pairs of cystein residues that form disulfide bridges and only one cystein residue that contains a free thiol group.

Activated protein aggregates can be prepared by various methods, such as heating, high pressure treatment etc. The resulting protein reactivity is determined by the overall treatment conditions (shear, protein concentration, type of protein, protein composition, type and concentration of salts, pH, other ingredients such as sugars and polysaccharides, fats). In order to be sufficiently reactive, the activated aggregates used in the preparation of the present encapsulates should exhibit a reactivity of at least 5.0 μmol thiol groups per gram, as determined in the Ellman's assay (Ellman, G. L. Tissue sulfhydryl groups. Arch. Biochem. Biophys. 1959, 82, 70-77).

The oxidative cross-linking of the free thiol groups in the activated protein aggregates is achieved with the help of suitable oxidizing agents. Examples of oxidizing agents that can suitable be employed include salts, oxides or ligands of transition metals and reactive oxygen compounds and oxidizing enzymes (oxidoreductases).

The present invention also encompasses encapsulates obtainable by the above mentioned method. The cross-linked protein-based encapsulates that can be obtained by the present method exhibit unique properties. The disulfide cross-linked protein-based encapsulation matrix can provide an extremely effective barrier against, for instance, moisture and oxygen.

### General Definitions

The term “encapsulate” as used herein refers to a particulate material. The individual particles within the encapsulate can consist of clearly identifiable discrete particles, but they can also consists, for instance, of a cluster of (micro-)particles, e.g. as a result of agglomeration.

“Probiotics” or “probiotic strain(s)” refers to strains of live micro-organisms, preferably bacteria, which have a beneficial effect on the host when ingested (e.g. enterally or by inhalation) by a subject.

The term “protein” as used herein refers to a polymer made of amino acids arranged in a chain and joined together by peptide bonds between the carboxyl and amino groups of adjacent amino acid residues. Typically, the protein contains at least 10 amino acid residues. The protein employed in accordance with the present invention can be, for instance, an intact naturally occuring protein, a protein hydrolysate or a synthesised protein.

The term “oxidizing agent” as used herein refers to a component that is capable of initiating formation of disulfide bridges between the present activated protein aggregates through the reaction of two or more thiol groups. The term “oil” as used herein encompasses any lipid substance that contains one or more fatty acid residues. Thus, the term oil encompasses, for instance, triglycerides, diglycerides, monoglycerides, free fatty acids and phospholipids. The oil employed in accordance with the present invention can be a solid, a liquid or a mixture of both.

The term “comprising” is to be interpreted as specifying the presence of the stated parts, steps or components, but does not exclude the presence of one or more additional parts, steps or components.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The term “sensitive components” encompasses components or ingredients which benefit from being protected from the environment (especially from the digestive tract or parts thereof, but also light, temperature, acids, radiation, etc.) and includes e.g. flavours, colourants, salts, enzymes, microorganisms (e.g. bacteria such as one or more probiotic bacterial strains), fibres, peptides, minerals, vitamins, oils, pharmaceutically active substances, bioactive components, hormones, gas, etc.

### DETAILED DESCRIPTION OF THE INVENTION

In one embodiment the invention provides a method of producing a protein-based encapsulate, said method comprising:
providing an aqueous solution of a protein that is capable of forming disulfide cross-links;
submitting said aqueous solution to a protein activation treatment to produce an aqueous suspension of activated protein aggregates, said suspension having a reactivity of at least 5.0 μmol thiol groups per gram of activated protein aggregates as determined in the Ellman's assay;
dispensing said aqueous suspension in a gas or a water-immiscible liquid to produce suspension droplets having a diameter of 0.1-500 μm; and
forming disulfide cross-links between the activated protein aggregates by contacting the activated protein aggregates with an oxidizing agent, optionally after said activated protein aggregates have been partially cross-linked by forming disulfide cross-links by means of heat treatment or by pressurization to a pressure in excess of 50 MPa.
   Optionally further layers are added around the microcapsules obtained by said method.

### Aqueous Solution of a Protein Capable of Forming Disulfide Cross-Links

In step a) of the method a protein, most preferably a food-grade protein is dissolved in an aqueous solution, such as for example water. Preferably whole (essentially intact/full-length) proteins are used, although in certain embodiments also peptides, or hydrolyzed or partially hydrolyzed proteins and/or peptides may be used. Suitable isolated proteins may be obtained from various sources. They may be extracted or purified from natural sources, such as plants, animal milk, animal tissue, microorganism, etc. using known methods or they may be obtained commercially. Suitable proteins or protein compositions (i.e. mixtures of different types of proteins and/or proteins from different sources) include for example total milk proteins, individual milk proteins, such as one or more whey proteins, e.g. β-lactoglobulin, α-lactalbumin, bovine serum albumin, etc., and/or one or more caseins such as α-caseins, α-caseins, κ-caseins and γ-caseins or total caseins or total whey proteins. Total whey proteins can for example be obtained from Davisco Foods, USA (e.g. BiPRO™).

Other suitable protein sources are plant proteins, such as one or more (e.g. total) wheat proteins, soy proteins, pea proteins, lupine proteins, canola or oilseeds rape proteins, maize proteins, rice proteins, and many others. Similarly, animal proteins, one or more blood proteins, such as bovine serum albumin, one or more egg, meat or fish-proteins may be used. In one embodiment also microbial proteins such as one or more bacterial proteins and/or fungal proteins (including yeast proteins) are used. It is understood that also recombinantly produced proteins may be used, such as e.g. recombinantly produced β-lactoglobulin.

In a preferred embodiment of the invention the protein that is capable of forming disulfide cross-links is selected from one or more of the group consisting of whey proteins, egg proteins, soy proteins, lupine proteins, rice proteins, pea proteins, wheat proteins and combinations thereof. Most preferably, said protein is a whey protein.

Especially preferred whey proteins for use in the method are one or more of whey protein isolate, whey protein concentrate, β-lactoglobulin and a mixture of β-lactoglobulin and α-lactalbumin.

In order to prepare a protein-based encapsulation matrix that exhibits a high level of disulfide cross-linking it is advisable to employ a protein containing at least three cross-linkable groups. Accordingly, in a preferred embodiment the protein that is capable of forming disulfide cross-links comprises at least three cystein residues per molecule, even more preferably at least 4 and most preferably at least 5 cystein residues per molecule. The whey proteins β-lactoglobulin and α-lactoglobulin contain 5 and 8 cystein residues per molecule, respectively. The term “cystein residue” also encompasses cystein residues that are bound to other cystein residues by means of a disulfide bond.

In one embodiment the proteins preferably comprises at least about 1 or even 2 cystein residues per 500, especially per 400 amino acids, more preferably at least 1 or even 2 cystein residues per 300 or 200 amino acids, even more preferably per 100, 30 or 20 amino acids. The average molecular weight of the protein is preferably at least 1, 5, 10, 15, 20, 50, 100, 200, 250 or more kDa as determined by SDS-PAGE analysis.

When protein hydrolysates are used, the hydrolysis is preferably such that at least 20%, 30%, more preferably at least 40 or 50% (or more, e.g. 60, 70, 80 or 90%) of the protein fragments in the hydrolysate have a length of at least about 10, 20 or 30 amino acids or longer, such as 40, 50, 60 amino acids or more.

In accordance with the invention it is preferred that the aqueous solution contains from 0.1-50 wt % of the protein that is capable of forming disulfide cross-links. More preferably said aqueous solution contains 0.2-25 wt %, most preferably 0.5-15 wt % of said protein. It should be understood that the aqueous solution of protein that is capable of forming disulfide cross-links can also contain non-dissolved protein and other non-dissolved components.

Depending on the exact type of encapsulate which is to be made, one or more additives may be added to (and mixed with) the aqueous protein solution either prior to protein activation and/or after protein activation, in the above method or may be added as such during the steps of dispensing the aqueous suspension in the gas or the water-immiscible liquid or during the process of contacting the activated protein aggregates with the oxidizing agent. Additives that may be suitably added are described further below.

In certain embodiments these additives include “sensitive components” that need to be protected from exposure to external factors and that are suitably incorporated in the present encapsulate. Also, in certain embodiments of the invention, further additives may be incorporated, e.g. additives that can be used to further modulate the release characteristics of the encapsulate, e.g. plasticizers and the like.

### Activation Treatment

In the present method, the protein solution (which optionally comprises further additives) is submitted to a protein activation treatment. The nature of this treatment is not essential, as long as the protein becomes sufficiently activated for further use. Thus, although the activation treatment is preferably a heat treatment, other methods are also suitable for achieving the same degree of protein activation, such as application of high pressure, shear forces, etc. Examples of suitable methods for achieving adequate protein reactivity are heat treatment, microwave treatment, exposure to very high pressure, application of shear, unfolding with urea, and combinations thereof. The skilled person can easily determine whether the treatment results in sufficiently activated (reactive) protein aggregates.

When heat treatment is used to activate the proteins, the temperature and time required for obtaining the minimum reactivity depends on the types of protein used and other conditions, such as applied shear, pH of the solution, salts, etc. For example, heat treatment of a solution of 9% wt. whey proteins (BiPRO™; Davisco, USA) in demineralized water for 30 minutes holding time at 90° C. in a water bath without stirring resulted in a reactivity of more than 15 μmol per gram of protein.

The activation treatment preferably comprises heating the aqueous solution to a temperature of at least 60° C. and less than 200° C. for at least a period of time equal to t, which period of heating t is governed by the following formula: t=(500/(T−59))−4 wherein: t=duration of heating (in seconds) and T=heating temperature (in ° C.). More preferably the heating conditions complied are governed by the following formula: t=(90000/(T−59))−900.

In a preferred embodiment of the invention, a method as defined herein before is provided, wherein the activated protein aggregates have a volume weighted average diameter in the range of 1-1000 nanometers, more preferably within the range of 2-250 nanometers, even more preferably within the range of 2-100 nanometers.

### Reactivity

Whatever treatment is used for activation, the treatment should be sufficient to yield protein aggregates having a reactivity of at least 5.0 μmol thiol groups per gram of activated protein aggregates. For example, whey protein dissolved in water was found to reach sufficient reactivity when exposed to 90° C. for 30 minutes, but other activation treatments may lead to similar reactivity.

Reactivity is required to covalently cross-link protein aggregates. The reactivity is defined as the number of thiol groups per amount of protein expressed as μmol thiol groups per gram of activated protein aggregates. Exposure of reactive thiol groups, which is a prerequisite for reactivity, can be achieved by e.g. heat-treatment.

In a particularly preferred embodiment of the invention, a method is provided as defined herein before, wherein the activated protein aggregates have a reactivity of at least 10 μmol, more preferably at least 15 μmol, even more preferably at least 20 μmol and most preferably of at least 25 μmol thiol groups per gram of activated protein aggregates.

### Ellman's Assay

Reactivity can be determined at pH 7 according to the Ellman's assay (Ellman, 1959 vide supra). In this assay the number of thiol groups is determined using ε(412 nm)=13,600 M⁻¹ cm⁻¹ for 2-nitro-5-mercaptobenzoic acid (DTNB) and expressed as the amount of thiol groups (μmol) per gram of protein (aggregates). The absorbance is measured at 20-25° C. The value after 30 minutes of incubation with DTNB is taken to calculate the reactivity. Hence, reactivity is determined after 30 minutes of incubation at 20-25° C. of a 2 wt % protein solution, using ε(412 nm)=13,600 M⁻¹ cm⁻¹ for 2-nitro-5-mercaptobenzoic acid (DTNB).

A convenient way to perform the Ellman's assay is described in Alting et al. (Formation of disulphide bonds in acid-induced gel of preheated whey protein isolate. J. Agric. Food Chem. 48 (2000) 5001-5007). Typically, 0.25 ml of a 1 mg/ml DTNB solution in 50 mM imidazol-buffer pH 7 (pH adjusted with HCl), 0.2 mL protein solution (2 wt % protein solution) and 2.55 ml imidazol-buffer pH 7 are mixed. The assay is preferably performed in the absence of detergents such as urea or SDS.

### Dispensing the Aqueous Suspension of Activated Protein Aggregates

In the present method, the aqueous suspension comprising the reactive protein aggregates (and optionally other additives) is advantageously dispensed in a gas or in a water-immiscible liquid to produce suspension droplets having a volume weighted average diameter in the range of 0.1-500 μm, more preferably in the range of 0.5-250 μm. The exact nature of the gas or water-immiscible liquid is not crucial provided that it allows for the formation of the suspension droplets. Preferably the gas or water-immiscible liquid has low or zero reactivity towards the thiol groups contained in the activated protein aggregates. Preferred examples of gases that may be used in accordance with the invention include nitrogen, carbon dioxide, air, argon, helium and combinations thereof. Most preferably said gas is selected from the group consisting of nitrogen and air.

Preferably, the water-immiscible liquids in accordance with the invention can be separated from the microcapsules formed in the present method by convenient and routine processing, e.g. by evaporation at moderately increased temperatures and/or moderately reduced pressure. It is also feasible to employ a water-immiscible liquid that is non-volatile (e.g. triglyceride oil) and to remove said liquid by means of solvent extraction, e.g. by using hexane or supercritical carbon dioxide as the extraction solvent. Preferred examples of water-immiscible liquids therefore include oil, hexane, supercritical fluids and combinations thereof. The suspension of activated protein aggregates is typically dispensed in the gas or water immiscible liquid by means of a nozzle.

The gas or liquid into which the suspension of protein aggregates is dispensed advantageously has a temperature in excess of 40° C., even more preferably in excess of 60° C. By subjecting the dispensed suspension to a substantial temperature increase initial cross-linking of the protein aggregates can be instigated. By partially cross-linking the protein aggregates in the suspension droplets the stability of these droplets is enhanced, which makes it easier to oxidatively cross-link the protein aggregates in the next step.

In a preferred embodiment the aqueous suspension is dispensed into a hot gas to remove water and to convert the droplets into partially cross-linked protein-based particles which are subsequently contacted with the oxidizing agent. Particularly good results are obtained if the dispensed suspension is contacted with the hot gas in countercurrent fashion. Furthermore, preferred embodiments of the invention provide a method as defined before, wherein suspension droplets are produced having a volume weighted average diameter within the range of 0.1-1000 μm, most preferably within the range of 0.5-250 μm.

### Forming Disulfide Cross-Links

In the present method, disulfide cross-links between the activated protein aggregates are formed by contacting the activated protein aggregates with an oxidizing agent. Optionally this step is preceded by heat treatment or pressurization to partially cross-link the activated protein aggregates by the formation of disulfide bonds.

The oxidizing agent according to the invention has the ability to oxidize the free thiol groups in the protein aggregates to form disulfide cross-links. Any oxidizing agent having this ability may be used in accordance with the invention. Preferably, the oxidizing agent is selected from the group consisting of salts, oxides or ligands of transition metals, reactive oxygen compounds (e.g. hydrogen peroxide) and oxidizing enzymes (oxidoreductases) and combinations thereof.

Preferred examples of transition metals that can be used in the form of oxidizing salts, oxidizing oxides or oxidizing ligands in the present method are selected from the group consisting of copper, iron, manganese, nickel, zinc, ruthenium, cobalt and combinations thereof. Most preferably, the transition metal is selected from the group consisting of copper, iron, manganese, zinc and combinations thereof. According to another preferred embodiment, the present method employs a salt or an oxide of a transition metal, e.g. a transition metal oxide or a transition metal halide. The term “salt” and “oxide” as used herein also encompasses the use of dissociated salts. Examples of transition metal salts and oxides that can suitably be employed in accordance with the present invention include CuSO₄, FeCl₃, CuCl₂, Na₃VO₄, Na₂MoO₃.

Furthermore, it is preferred that the protein aggregates are contacted with the one or more transition metals in an aqueous medium containing at least 0.001 mM of the said transition metals, more preferably 0.001-500 mM, most preferably 0.01-100 mM. Preferably, said transition metals are contained in the aqueous medium in the form of cations having a valency of at least 2.

Oxidoreductases (i.e. enzymes classified under the Enzyme Classification number E.C. 1 (Oxidoreductases) in accordance with the Recommendations (1992) of the Interantional Union of Biochemistry and Molecular Biology (IUBMB)) are enzymes catalyzing redox reaction. Suitable examples include laccases or related enzymes which act on molecular oxygen and yield water; oxidases, which act on molecular oxygen and yield peroxide; and peroxidases which act on peroxide and yield water. Hence, in a preferred embodiment of the invention, a method is provided as defined herein before, wherein the oxidizing agent is an enzyme selected from the group consisting of oxidases, peroxidases, laccases and combinations thereof. More preferably the oxidizing enzyme is selected from the group consisting of glutathione peroxidase, horseradish peroxidase, microperoxidase, coprinus cinereus oxidase, chloroperoxidase, lactoperoxidase, manganese peroxidase and combinations thereof. Most preferably, the oxidizing enzyme is selected from the group consisting of glutathione peroxidase, horseradish peroxidase, coprinus cinereus oxidase, manganese peroxidase and combinations thereof.

Examples of reactive oxygen substances that can suitably be employed include hydrogen peroxide, alkyl hydroperoxides and dialkyl peroxides, hydrogen peroxide being most preferred.

In a preferred embodiment of the invention, a method as defined herein before is provided, wherein prior to or concurrent with the contacting of the activated protein aggregates with the oxidizing agent, the method comprises the step of forming disulfide cross-links between the activated protein aggregates by heating the suspension droplets to a temperature of a least 40° C. for at least 5 milliseconds and/or by pressurizing the suspension droplets to a pressure of at least 50 MPa. More preferably said step comprises heating the suspension droplets to a temperature within the range of 50-150° C., most preferably within the range of 60-120° C., preferably for 1-86,000 seconds, more preferably for 20-86,000 seconds.

Cross-linking by pressurization preferably involves pressures within the range of 50-1000 MPa, most preferably within the ranges of 100-600 MPa. Said pressures may typically be applied for at least 0.1 second, preferably for 1-7200 seconds.

Without wishing to be bound by theory, it is hypothesized that after cross-linking by heat treatment or pressurization some free thiol groups remain in the cross-linked matrix. The presence of these free thiol groups may allow for rearrangements of the disulfide cross-links to occur. In the present treatment with oxidizing agent the accessible thiol groups present are readily oxidized, thus preventing such rearrangements from occurring, and, very likely, additional disulfide cross-links are formed, thus further strengthening the protein network. These effects may well account for the extraordinary properties of the present protein-based encapsulates.

In accordance with a particularly preferred embodiment the level of cross-linking in the protein-based encapsulation matrix is sufficiently high to render it sufficiently acid resistant to ensure that the encapsulate remains intact in the stomach so that the encapsulated component (s) are only released when contacted with enzymes secreted into the lower intestinal tract, such as pancreatic enzymes.

### Components to be Encapsulated

The present method is suitably used for encapsulating sensitive components, as noted before. “Sensitive components”, according to this invention, include any ingredient benefiting from being protected from the environment (especially from the digestive tract or parts thereof, but also light, temperature, acids, radiation, etc.) and include e.g. flavours, colourants, polyphenols, enzymes, micro-organisms (e.g. bacteria such as one or more probiotic bacterial strains), fibres, peptides, minerals, vitamins, fatty acids (e.g. PUFAs), pharmaceutically active substances, bioactive components, hormones etc. However, this list is non-limiting, as any component, preferably food-grade, which benefits from protection against the environment, such as oxygen, moisture, acid conditions, interaction with food matrix, temperature, any part of the intestinal tract environment (e.g. mouth/saliva, stomach acids, intestine, etc.) etc. may be used as well as any other component that is to be separated from its environment simply to prevent the escape thereof, e.g. volatile components as well as gases, in particular air. In a preferred embodiment of the invention, a method as defined herein before is thus provided, wherein a component is encapsulated, said component being selected from the group consisting of enzymes, micro-organisms, vitamins, minerals, peptides, polyphenols, fatty acids, oils, pharmaceutically active substances, bioactive components, flavours, colourants, fibres, gas and combinations thereof.

Preferably the component to be encapsulated is not reactive towards the activated protein aggregates, e.g. the component does not react with free thiol groups as this would interfere with the cross-linking of the protein in the subsequent step(s).

In one embodiment of the present invention a method is provided as defined herein before, wherein the component to be encapsulated is dissolved in, or homogeneously dispersed throughout the suspension of activated protein aggregates. This method will typically yield encapsulates wherein the component is evenly distributed throughout the cross-linked protein matrix.

In accordance with a particularly preferred embodiment of this invention a fat or fat-containing material is added to the aqueous protein containing system, either before or after the activation treatment, to form an oil-in-water emulsion, which is than dispensed into the gas or water-immiscible liquid as described herein before. The fat or fat-containing material may itself constitute (part of) a sensitive component to be encapsulated, e.g. when the fat is rich in polyunsaturated fatty acids (PUPA), in particular fats or oils comprising or consisting of omega-3 and/or omega-6 fatty acids. Alternatively, the fat may serve as a carrier or solvent for a fat-soluble sensitive component.

In another embodiment of the present invention the component to be encapsulated is a gas. Typically, in accordance with this embodiment of the invention the aqueous suspension containing the activated protein aggregates is dispensed in a gas to form droplets containing gas bubbles which are subsequently contacted with an oxidizing agent as described herein before. Preferably the suspension is dispensed in the gas using a spray drying apparatus. This type of processing can be carried out in accordance with methods known in the art, e.g. as described in U.S. Pat. No. 6,223,455 or the “Spray Drying Handbook”, K. Masters, 5^{th} ed., Longman Scientific & Technical Publishers, 1991, pp. 329-337 and 346-349.

In accordance with another embodiment, the present method comprises spraying the suspension of protein aggregates onto core particles, e.g. in a fluidized bed, said core particles typically (though not necessarily) containing the component(s) to be encapsulated. Typically, in accordance with this embodiment of the invention, the core particles are suspended in the same gas into which the suspension is dispersed. Thus, the suspension droplets are deposited on the surfaces of the core particles. The protein aggregates deposited on the surface of the core particles may cross-linked as soon as they have been deposited onto the core particles, e.g. by applying heat treatment or by applying core particles that contain a suitable oxidizing agent. Alternatively, cross-linking may take place after a suitable layer of activated protein aggregates has been deposited.

Preferably, the activated protein aggregate suspension is sprayed onto the core particles and dried using e.g. fluidized bed or spouted bed equipment. Such equipment is available in the art, see e.g. Fluid bed coater GPCG 1.1 with Wurster insert (Glatt GmbH).

In accordance with a preferred embodiment, the core particles comprise at least 10 wt. %, more preferably 10-98 wt %, most preferably 50-98 wt % of a bulk ingredient. Various bulk ingredients may be used in accordance with the invention. For example, the bulk ingredient may comprise or consist of hydrocolloids (e.g. carboxymethylcellulose, starch, maltodextrin) and/or fats and/or waxes and/or carbohydrates (e.g. sugars) and/or proteins.

Preferably said core particles further comprise one or more of the components selected from the group consisting of enzymes, micro-organisms, fibres, vitamins, minerals, peptides, polyphenols, fatty acids, oils, pharmaceutically active substances, bioactive components, flavours, colourants, gas and combinations thereof. One or more of the components can be entrapped within the core particle made by e.g. extrusion or other technique. Preferably the sensitive component(s) are either entrapped within the core particle material or coated onto the core particle. In another, less preferred embodiment they contained in the suspension of activated protein aggregates that is applied onto the core particles.

The core particles are preferably spherical. Suitable core particles include particles of at least 50 μm. Preferably the core particles have a diameter of at least 100 μm even more preferably of at least 200 μm and most preferably of at least 300 μm. Typically, the diameter of the core particles does not exceed 5000 μm.

### Additives

In one embodiment one or more further additives (e.g. fats, hydrocolloids, carbohydrates, protein, etc.) are added to the protein aggregates either before, during or after protein activation, but prior to dispension of the aqueous suspension in the gas or the water-immiscible liquid. In another embodiment these additives are coated onto the encapsulates, typically after the oxidative cross-linking.

Typically, one or more of the following (food-grade) additives may be added to the protein aggregates:
humectants, in particular polyols such as: glycerol, xylitol;
plasticizers, such as glycerol, glyceryl triacetate and/or di-(2-ethylhexyl) adipate, or others, or mixtures of two or more plasticizers; the addition of one or more plasticizers improves the flexibility of the protein coating; a preferred plasticizer is e.g. glycerol; the plasticizer is preferably added to the activated protein aggregates and mixed in an amount of 10 to 70 wt % on the protein basis, most preferably 20 to 40 wt %.
sugars such as for example: lactose, sucrose, glucose, galactose
hydrocolloids such as for example: gum Arabic, alginate, pectin, starch, xanthan gum, carrageenan, guar gum, locust bean gum, tara gum, gellan gum.
salts such as for example: sodium salts, calcium salts, potassium salts;
cross-linkers such as for example: tannins, transglutaminase, formaldehyde, glutaraldehyde;
fats, in particular food-grade fats, such as plant derived oil (e.g. sunflower oil, canola oil, palm oil, soybean oil, flax oil, safflower oil, peanut oil, maize oil, olive oil, pumpkin oil, etc.);
waxes; and
proteins, such as gelatin.

Preferably the additives are not reactive towards the activated protein aggregates, e.g. the additives do not react with free thiol groups as this would interfere with the cross-linking of the protein in the subsequent spray step. The exception to this concerns cross-linkers which will assist in crosslinking the activated protein aggregates, hence cross-linkers preferably are susceptible to reaction with sulfur groups.

### Coating Layers

The encapsulates formed in this method may be used as such or they may be coated with one or more coating layers. For example, to add further coatings, the microcapsules contained in the encapsulate may be used as “core” particles. Optionally, one or more further layer s of activated protein aggregate and/or one or more of the above-defined sensitive components and/or further additives, can be applied on the encapsulates obtainable by the present method to create multi layered encapsulate particles. Any suitable coating method may be used for the addition of further layers, such as spray drying drum drying fluidized bed coating, etc. Optionally, spray drying can occur in the presence of modified atmosphere, N₂, or other gas for additional protection of the sensitive ingredient.

Single or multi-layered encapsulates in accordance with the invention preferably have a diameter of at least 100 μm, more preferably of at least 250 μm and most preferably of at least 400 μm. Preferably, these coated particles have a volume weighted averaged diameter in the range of 200-5000 μm, preferably in the range of 300-2000 μm. Size and shape can be analyzed using microscopy (e.g. light microscopy or electron microscopy) or light scattering.

### Protein Encapsulates

Another aspect of the invention relates to an encapsulate obtainable by the method as defined herein before, said encapsulate comprising a protein-based encapsulation matrix that envelops one or more actives selected from the group consisting of enzymes, micro-organisms, fibres, vitamins, minerals, peptides, polyphenols, fatty acids, oils, pharmaceutically substances, bioactive components, flavours, colourants, gas and combinations thereof and combinations thereof.

The activation treatment and the cross-linking step(s) of the method of the present invention all provide means, independent of another, for control ling the water-solubility of the encapsulates. For many applications it is preferred that the encapsulates are largely water-insoluble.

According to a particularly preferred embodiment, the encapsulates are characterized in that less than 75 wt. %, preferably less than 40 wt. % of the protein contained in the protein-based matrix can be dissolved when 75 mg of the encapsulate is dispersed in 50 ml distilled water having a temperature of 5° C. at any pH within the range of 3.0-7.0.

According to an even more preferred embodiment the weight percentage of the protein that can be dissolved is at least a factor 1.3 higher when in the aforementioned procedure under the distilled water is replaced by an aqueous solution of 2 wt. % dithiothreitol (DTT).

In the above mentioned solubility tests and the solubility tests described elsewhere in this document the pH of the distilled water or the DTT solution is adjusted with the help of HCl and solubility is measured 16 hours after the encapsulate was dispersed in the liquid. During this period the mixture is continuously gently stirred in order to prevent ‘clumping’ of the encapsulate particles. In both the solubility test i) and ii) pH is adjusted to achieve maximum protein solubility within the pH range of 3.0-7.0.

The poor solubility of the cross-linked protein-based matrix in distilled water is indicative for the high level of cross-linking Without the disulfide cross-links the protein-based matrix of the present encapsulate would exhibit a much higher water solubility. This can be demonstrated by repeating the solubility test i) using an aqueous dithiothreitol (DTT) solution instead of distilled water. Since DTT reduces disulfide bonds and maintains the monothiols in a reduced state, the difference in solubility observed in the solubility tests with the DTT solution and distilled water is indicative of the level of disulfide cross-linking

According to a very preferred embodiment, the protein-based matrix is characterized in that more than 50 wt. %, more preferably more than 60 wt. %, even more preferably more than 80 wt. % and most preferably at least 90 wt. % of the protein contained in the protein-based matrix dissolves in an aqueous solution of 2 wt. % DTT having a temperature of 25° C. and a pH in the range of 3.0-7.0.

In accordance with another preferred embodiment less than 40 wt. %, more preferably less than 25 wt. % of the protein contained in the protein-based matrix can be dissolved when 75 mg of the encapsulate is dispersed in 50 ml distilled water having a temperature of 25° C. at any pH within the range of 1.0-8.0.

According to another advantageous embodiment the present encapsulate is not soluble under conditions prevailing in the human stomach. Thus, most preferably, less than 50 wt. %, more preferably less than 40 wt. % and most preferably less than 30 wt. % of the protein contained within the protein-based encapsulation matrix dissolves when 75 mg of the encapsulate is dispersed in 50 ml of aqueous HCl solution with pH 3.0 under continuous stirring for 8 hours, at a temperature of 37° C. Naturally, the stirring conditions employed in the above tests should be gentle, i.e. sufficient to disperse the encapsulate and not to mechanically break up the protein microcapsules, typically they should be sufficient to simulate the shear forces resulting from gastric movement.

The encapsulates of the present invention contain a protein-based matrix that is made up of macromolecules consisting of a hundreds or thousands of protein molecules that have been cross-linked by disulfide bonds. According to a particularly preferred embodiment, the protein that has been cross-linked by disulfide cross-links exhibits a number weighted average degree of polymerisation of at least 500 more preferably of at least and most preferably of at least 1000. Here the degree of polymerisation equals the total number of protein molecules that are linked together in a single cross-linked macromolecule.

The encapsulates of the present invention may advantageously be employed as a vehicle for delivering biologically active ingredients to an animal or a human. In particular protein microcapsules that are stable under gastric conditions may suitably be used to deliver biologically active ingredients that are not stable under gastric conditions. Thus, one aspect of the invention relates to the use of the present encapsulate in therapeutic or prophylactic treatment, said treatment comprising oral administration of the encapsulate. Typically, the protein microcapsules are orally administered in an amount of 0.1 to 40 g per administration event. In accordance with this aspect of the invention, the biologically active ingredient may be a pharmaceutically active ingredient or a nutrient (including micronutrients such as vitamins).

### Applications of the Encapsulates

Yet another aspect of the invention concerns the application of the present encapsulates in foodstuffs, beverages, nutritional supplements, cosmetic products, pharmaceutical products and animal feed.

Foodstuffs and beverages comprising the encapsulates include for example the following: cold or warm drinks, such as coffee, chocolate, tea, fruit or vegetable juices; soups; sauces; spreads, batters, ready-to-eat meals, dairy products (milk, milk-based drinks, yoghurt, cheese, butter, margarine, ice cream), pasta, fruit or vegetable products, meat or fish products, meat replacers, bread, pastries, deserts, sweets, candy-bars, confectionary, food- or drink-additives (such as coffee or tea creamers, sweeteners), powders such as instant coffee or tea, milk-powder, soup powder, ice-cream, etc.

Suitable amounts of the encapsulates may vary, depending on the product in which the encapsulate is applied. Typically, the encapsulate is applied in a concentration of at least 0.01 wt. %, preferably of at least 0.1 wt. % and most preferably of at least 0.3 wt. %. Usually, the amount in which the encapsulate is employed does not exceed 50 wt. %, more preferably it does not exceed 20 wt. % and most preferably it does not exceed 10 wt. %.

Yet, another aspect of the invention concerns a process of preparing a foodstuff, a beverage, a nutritional supplement, a cosmetic product, a pharmaceutical product or animal feed, said method comprising incorporating from 0.01-50 wt. %, more preferably 0.1-30 wt %, most preferably 0.3-10 wt % of an encapsulate as defined herein before.

The invention is further illustrated by means of the following examples.

### EXAMPLES

### Example 1

A protein solution was prepared by mixing 54 g of whey protein isolate (BiPRO™; Davisco, USA) in 546 g of demineralized water at room temperature (stirred for 2 h).

Reactive protein aggregates were prepared by heating the whey protein isolate solution at 90° C. during 7 minutes under shear in a heat exchanger. The solution was further cooled down in ice and then brought to room temperature. The reactivity of the particles was determined using the DTNB-method as described before. The reactivity was about 18 μmol thiol groups per gram protein.

The reactive protein aggregates were sprayed using a fluidized bed coater (Glatt, Germany) onto methylcellulose round core material (Cellets®, Syntapharm, Germany) with a diameter of 350 μm.

Next, the encapsulate so obtained was divided in 4 different portions of each 75 gram. These portions were dispersed in 4 different aqueous systems (50 ml) having a temperature of 20° C. and a pH of 7. The composition of these aqueous systems was as follows:
distilled water
10 mM FeCl₃.6H₂O
10 mM CuSO₄.5H₂O
10 mM H₂O₂

The capsules were gently stirred overnight. In the case of distilled water and the aqueous solution of Fe(III) and Cu(II), the supernatant was filtered and colored with BSA protein essay kit. The soluble proteins were quantified by spectrophotometer reading at 562 nm. In the case of the aqueous solution of H₂O₂, the supernatant was filtered and the soluble proteins were quantified by spectrophotometer reading at 280 nm. The solubility of the encapsulates was normalised to the solubility of the encapsulates in water of pH 7.

As shown in Table 2, the solubility of the encapsulates decreased as a result of contacting the encapsulates with an oxidizing agent. Thus, it can be concluded that additional cross-linking of the encapsulates prepared with reactive protein aggregates decreases the solubility of the encapsulates.

TABLE 2
Solubility of the encapsulates in the presence of oxidizing agents

| Solution | Relative solubility (%) |
|---|---|
| Distilled water | 100 |
| FeCl₃•6H₂O | 27 |
| CuSO₄•5H₂O | 1 |
| H₂O₂ | 63 |

1.-17. (canceled)

18. A method of producing a protein-based encapsulate, said method comprising:
(a) providing an aqueous solution of a protein that is capable of forming disulfide cross-links;
(b) submitting said aqueous solution to a protein activation treatment to produce an aqueous suspension of activated protein aggregates, said suspension having a reactivity of at least 5.0 μmol thiol groups per gram of activated protein aggregates as determined in an Ellman's assay;
(c) dispensing said aqueous suspension in a gas or a water-immiscible liquid to produce suspension droplets having a diameter of 0.1-500 μm; and
(d) forming disulfide cross-links between the activated protein aggregates by contacting the activated protein aggregates with an oxidizing agent.

19. The method according to claim 18, further comprising partially cross-linking said activated protein aggregates by forming disulfide cross-links by means of heat treatment or by pressurization to a pressure in excess of 50 MPa.

20. The method according to claim 18, wherein the oxidizing agent is selected from the group consisting of salts, oxides or ligands of transition metals, reactive oxygen compounds, oxidizing enzymes and combinations thereof.

21. The method according to claim 20, wherein the transition metal is selected from the group consisting of copper, iron, manganese, nickel, zinc, ruthenium, cobalt and combinations thereof.

22. The method according to claim 20, wherein the reactive oxygen compound is hydrogen peroxide.

23. The method according to claim 20, wherein the aqueous medium contains at least 0.001 mM of said transition metals.

24. The method according to claim 20, wherein the oxidizing enzyme is selected from the group consisting of oxidases, peroxidases, laccases and combinations thereof.

25. The method according to claim 18, further comprising dissolving in, or homogeneously dispersed throughout the suspension of activated protein aggregates a component to be encapsulated.

26. The method according to claim 24, wherein the component to be encapsulated is selected from the group consisting of enzymes, micro-organisms, vitamins, minerals, peptides, polyphenols, fatty acids, oils, pharmaceutically active substances, bioactive components, flavours, colourants, fibres, gas and combinations thereof and combinations thereof.

27. The method according to claim 18, wherein the droplets are formed by dispensing the aqueous suspension in a gas.

28. The method according to claim 18, wherein the aqueous solution contains from 0.1-25 wt. % of the protein that is capable of forming disulfide cross-links.

29. The method according to claim 18, wherein the activated protein aggregates have a reactivity of at least 10 μmol.

30. The method according to claim 29, wherein the activated protein aggregates have a reactivity of at least 15 μmol thiol groups per gram of activated protein aggregates.

31. The method according to claim 18, wherein the protein that is capable of forming disulfide cross-links comprises at least three cystein residues per molecule.

32. The method according to claim 18, wherein the protein that is capable of forming disulfide cross-links is selected from one or more of the group consisting of whey proteins, egg proteins, soy proteins, lupine proteins, rice proteins, pea proteins, wheat proteins and combinations thereof.

33. An encapsulate obtained by a method according to claim 18, said encapsulate comprising a protein-based encapsulation matrix that envelops one or more actives selected from the group consisting of enzymes, micro-organisms, fibres, vitamins, minerals, peptides, polyphenols, fatty acids, oils, pharmaceutically active substances, bioactive components, flavours, colourants, gas and combinations thereof and combinations thereof.

34. The encapsulate according to claim 33, wherein less than 50 wt. % of the protein contained within the protein-based encapsulation matrix is dissolved when 75 mg of the encapsulate is dispersed in 50 ml distilled water having a temperature of 5° C. at any pH in the range of 3.0-7.0.

35. A foodstuff, a beverage, a nutritional supplement, a cosmetic product, a pharmaceutical product or animal feed containing from 0.01-50 wt. % of an encapsulate according to claim 33.

36. A process of preparing a foodstuff, a beverage, a nutritional supplement, a cosmetic product, a pharmaceutical product or animal feed, said process comprising incorporating from 0.01-50 wt. % of an encapsulate according to claim 32 into the foodstuff, beverage, nutritional supplement, cosmetic product, pharmaceutical product or animal feed.
